(19) United States
(12) Patent Application Publication
(10) Pub. No.: US 2015/0245828 A1
(43) Pub. Date: Sep. 3, 2015

(54) TISSUE RETRACTOR

(71) Applicant: LapSpace Medical Ltd., Misgav Business Park (IL)

(72) Inventors: Shahar HARARI, Tel-Aviv (IL); Gilad LAVI, Rishon-LeZion (IL); Assaf LIVNE, Tel-Aviv (IL)

(21) Appl. No.: 14/635,073

(22) Filed: Mar. 2, 2015

Related U.S. Application Data
(60) Provisional application No. 61/946,817, filed on Mar. 2, 2014.

(51) Int. Cl. A61B 17/02

(52) U.S. Cl. CPC A61B 17/0218; A61B 2017/0225

(57) ABSTRACT

A tissue retractor device is provided. The device includes an inflatable tissue retractor head being attached to a distal end of a shaft having a conduit extending from a proximal end of said shaft to the inflatable tissue retractor; the inflatable tissue retractor head is optionally covered by a delivery sheath when deflated. The device also includes a handle which is detachably attached to the proximal end of the shaft. The handle includes a port for communicating an inflation fluid through the handle and the conduit to the inflatable tissue retractor when the handle is attached to the proximal end of the shaft. In some embodiments, the retractor device comprises a tensile element slidably attached to the inflatable head, the tensile element configured for controlling and/or maintaining an angulation of the inflatable head with respect to the shaft.

### RELATED APPLICATION

This application claims the benefit of priority under 35 USC 119(e) of U.S. Provisional Patent Application No. 61/946,817 filed on Mar. 2, 2014, the contents of which are incorporated herein by reference in their entirety.

### FIELD AND BACKGROUND OF THE INVENTION

The present invention relates to a tissue retractor configured for delivery through a narrow tissue port and to methods of delivering same through the port.

Minimally invasive surgery (MIS) is a surgical technique in which a body cavity (e.g. abdominal cavity) is accessed via several small incisions as opposed to the large incision access used in open surgery.

Surgical tools are inserted into the body cavity through ports positioned within the small incisions with the actuating handles of the surgical tools positioned outside the body. The surgeon manipulates the surgical tool via the handle while viewing the operative field on a video feed provided by a camera and a light source mounted on a rod inserted through one of the ports.

During minimally invasive surgery, the surgeon is required to expose and handle delicate tissues deep within the body cavities. This requires creation and maintenance of a surgical workspace large enough to enable the surgeon to view and work within the treatment area without damaging surrounding tissues.

To provide the surgeon with a good view of the operative field, the body cavity is usually insufflated with carbon dioxide gas and organs that obstruct the field of view of the camera and block access to the treatment area are retracted using a tissue retractor.

Tissue retractors are generally inserted into the body cavity in a collapsed conformation through an additional port, and are then expanded within the body and either held by an assistant or fixed to an object such as the operating table.

Tissue retractors known in the art typically utilize mechanically deployable arms/fingers (the Endo Retract™ by BioMedicon), baskets (A-Lap by EZSurgical) or hooks (Virtual Ports) which can be used to sweep and/or move tissue organs out of the treatment area. Inflatable retractors are also known in the art (e.g. Extrahand™ Balloon Retractor by BioMedicon), however due to their simple paddle-like configuration such retractors are more suitable for containing tissue than retracting it.

Trocar ports typically used for positioning of surgical tools and tissue retractors within a body cavity range from 5 mm to 12 mm in diameter. In recent years efforts to further minimize the impact of minimally invasive surgery on wall tissue have lead to an increase in use of 5 mm trocar ports. Although such ports substantially minimize surgical trauma to abdominal wall tissue and improve tissue access site closure following surgery, they limit the size of a laparoscopic tool head that can be delivered therethrough into the abdominal cavity.

Several laparoscopic tool configurations have been developed in order to traverse such limitations of 5 mm trocar ports. For example, U.S. Patent Application Publication No. 20100298774 describes an approach in which the laparoscopic tool shaft is delivered into a body cavity through a narrow working port (e.g. 5 mm) and back out of the body cavity through a wide access port (e.g. 10 mm) The working head is then attached to the distal end of the shaft (positioned outside the body) and is pulled back into the cavity through the wide access port. Although such an approach enables use of a narrow port for surgical access during surgery, it requires a second wide access port for attaching the working head to the shaft and requires a camera port for enabling such attachment.

While reducing the present invention to practice, the present inventors have devised novel approaches for delivering a tissue retractor having an inflatable working head into a body cavity through a narrow (e.g. 5 mm) trocar port.

### SUMMARY OF THE INVENTION

According to an aspect of some embodiments of the invention, there is provided a tissue retractor device comprising: an inflatable tissue retractor head attached to a distal end of a shaft, the inflatable retractor head comprising one or more finger-like extensions shaped and sized to move tissue within a body cavity, and a tensile element coupled to the inflatable tissue retractor head, the tensile element slidably received within one or more recesses configured in the head.

According to some embodiments of the invention, the tensile element is configured to resist bending of the head relative to the shaft from a set position.

According to some embodiments of the invention, the set position comprises an angulated position of the head relative to the shaft.

According to some embodiments of the invention, an angle between the head and the shaft is between 20-180 degrees.

According to some embodiments of the invention, the tensile element controls angulation of the head relative to the shaft.

According to some embodiments of the invention, the one or more recesses of the head are located at one or more webbing portions connecting between the finger-like extensions, and the tensile element is threadedly received within the one or more recesses.

According to some embodiments of the invention, the tensile element extends between the head and the shaft.

According to some embodiments of the invention, the tensile element is attached to the shaft by a hook like element, the hook like element axially moveable within the shaft.

According to some embodiments of the invention, each end of the tensile element is attached to a separate hook like element, the elements axially movable with respect to the shaft and with respect to each other to articulate the head.

According to some embodiments of the invention, the tensile element is configured to slide with respect to an outer surface of the head to a limited extent that reduces a risk of tear to the head when inflated, while still providing for control of angulation using the tensile element.

According to some embodiments of the invention, pulling on the tensile element reduces the angle between the head and the shaft, and loosening the tensile element allows the angle to increase.

According to some embodiments of the invention, the tensile element is strong enough to resist a natural urge of the head, when inflated, to extend linearly with respect to the shaft.

According to some embodiments of the invention, the set position comprises a linear position of the head with respect the shaft, in which the head is positioned substantially at a 180 degree angle relative to the shaft.

According to some embodiments of the invention, the tensile element is in the form of a strap, string, cable or band.

According to some embodiments of the invention, the tensile element is wrapped around at least one finger-like extension, forming a lasso-like configuration.

According to some embodiments of the invention, the head comprises two recesses through which the tensile element extends, the recesses symmetrically arranged with respect to the head.

According to some embodiments of the invention, the tensile element is external to the inflatable retractor head.

According to some embodiments of the invention, the device further comprises a sheath shaped and sized to cover the inflatable head in a deflated state, the sheath shaped and sized to slidably fit within a lumen of a trocar.

According to an aspect of some embodiments of the invention, there is provided a tissue retractor device comprising: an inflatable tissue retractor head attached to a distal end of a shaft, the inflatable retractor head comprising one or more finger-like extensions shaped and sized to move tissue within a body cavity, and a tensile element coupled to the inflatable tissue retractor head, the tensile element resisting bending of the inflatable tissue retractor head relative to the shaft to maintain the head at a substantially linear configuration relative to the shaft.

According to one aspect of the present invention there is provided a tissue retractor device comprising: (a) an inflatable tissue retractor head being attached to a distal end of a shaft having a conduit extending from a proximal end of the shaft to the inflatable tissue retractor; and (b) a handle being detachably attached to the proximal end of the shaft, the handle including a port for communicating an inflation fluid through the handle and the conduit to the inflatable tissue retractor when the handle is attached to the proximal end of the shaft.

According to further features in preferred embodiments of the invention described below, the port is in fluid communication with a lumen in the handle, the lumen being capable of sealing with the conduit when the handle is attached to the proximal end of the shaft.

According to still further features in the described preferred embodiments the inflatable tissue retractor head is configured such that when inflated with a fluid, the tissue retractor head forms a configuration which includes a plurality of finger-like extensions.

According to still further features in the described preferred embodiments the device further comprises a mechanism for angling a portion of the tissue retractor head with respect to the handle during or following inflation of the tissue retractor head.

According to still further features in the described preferred embodiments the device further comprises a sheath for covering the tissue retractor head when deflated, the cover being configured for delivery through a tissue incision.

According to still further features in the described preferred embodiments the sheath is axially rigid to enable penetration through the incision and radially flexible to enable covering of inflatable tissue retractor head when deflated.

According to another aspect of the present invention there is provided a tissue retractor device comprising: (a) an inflatable tissue retractor head being attached to handle through a shaft; and (b) a sheath for covering the tissue retractor head when deflated, the sheath being configured for delivery through an incision.

According to still further features in the described preferred embodiments the sheath is axially rigid to enable penetration through the incision and radially flexible to enable covering of inflatable tissue retractor head when deflated.

According to still further features in the described preferred embodiments the sheath includes a protrusion which functions as a stop for preventing the sheath from completely going through the incision and into the cavity.

According to another aspect of the present invention there is provided a method of positioning a laparoscopic device in a body cavity, the method comprising: (a) delivering a working head of the laparoscopic device through an incision and into the body cavity using a device shaft attached thereto; (b) sliding a trocar over the shaft; and (c) attaching a handle of the laparoscopic device to the shaft.

According to still further features in the described preferred embodiments the method further comprises inserting the trocar into the incision following (b) or (c).

According to still further features in the described preferred embodiments the working head is an inflatable tissue retractor head.

According to still further features in the described preferred embodiments (a) is effected by packing the tissue retractor head into a sheath configured for delivery through the incision.

According to still further features in the described preferred embodiments the sheath is axially rigid to enable penetration through the incision and radially flexible to enable covering of inflatable tissue retractor head when deflated.

According to still further features in the described preferred embodiments the method further comprises: (d) removing the sheath and inflating the inflatable tissue retractor head from the handle.

The present invention successfully addresses the shortcomings of the presently known configurations by providing a surgical device which can be delivered through a single narrow tissue access port.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A illustrates the present tissue retractor with the shaft disconnected from the handle, the sheath covering the inflatable retractor head and the trocar port prior to mounting on the shaft;

FIG. 1B illustrates the present tissue retractor with the shaft disconnected from the handle, the sheath covering the inflatable retractor head and the trocar port mounted on the shaft;

FIG. 1C illustrates the present tissue retractor with the shaft connected to the handle, the sheath removed from the inflatable retractor head and retracted proximally along the shaft and the trocar port mounted on the shaft and juxtaposed against the handle;

FIG. 1D illustrates the present tissue retractor with the shaft connected to the handle, the sheath removed from the shaft and the trocar port mounted on the shaft and juxtaposed against the handle;

FIG. 1E illustrates the present tissue retractor with the inflatable retractor head deployed and the trocar port mounted on the shaft and juxtaposed against the handle;

FIG. 2 illustrates the connector region between handle and shaft of the present tissue retractor;

FIGS. 3A-B illustrate the sheath (FIG. 3A), and the sheath and folded inflatable retractor head (FIG. 3B);

FIGS. 4A-B illustrate the inflation fluid female connector disposed within the handle and the male connector of the shaft;

FIGS. 5A-E illustrate delivery of the present tissue retractor into a body cavity through a narrow trocar port;

FIG. 6 illustrates the present tissue retractor with the inflatable retractor head positioned in a linear configuration with respect to the shaft, according to some embodiments;

FIG. 7 illustrates the present tissue retractor with the inflatable retractor head positioned in a linear configuration with respect to the shaft, shown from an opposite direction to FIG. 6, according to some embodiments;

FIGS. 8A-B illustrate the present tissue retractor with the inflatable retractor head positioned at an angle with respect to the shaft, according to some embodiments;

FIGS. 9A-B illustrate an exemplary attachment between a tensile element and the shaft of the tissue retractor device, according to some embodiments; and

FIGS. 10A-C illustrate the present tissue retractor comprising a movable sheath, according to some embodiments of the invention.

### DESCRIPTION OF THE PREFERRED EMBODIMENTS

The present invention is of a tissue retractor which can be delivered through a narrow access port (e.g. 5 mm trocar) and used in a laparoscopic surgical procedure to grasp, move and contain tissue organs.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In a previously filed patent application, the present inventors described a tissue retractor that can be effectively used for grasping and moving tissue as well as containing it while minimizing trauma to the organ and surrounding tissue. The tissue retractor incorporates an inflatable tissue retraction head (e.g. non-compliant balloon) which is configured for selectively engaging, grasping and containing tissue organs. Such selective modes of operation are controlled by the degree of deployment of the tissue retractor head which is in turn controlled by inflation and/or by mechanical elements.

While delivery of this tissue retractor through a wide trocar port (e.g. 10-12 mm) can be effected with ease, delivery through narrow trocar ports (e.g. 5 mm) can be challenging due to the diameter of the deflated and folded tissue retractor head.

In order to overcome such limitations, the present inventors devised a tissue retractor configuration that can be easily delivered through narrow ports and yet retains the tissue retraction area of the previously described inflatable retractor head and its functionality.

Two novel features of the present tissue retractor facilitate delivery of a tissue retractor having an inflatable retractor head through a narrow access port, a detachable handle with a sealable fluid conduit and a sheath for covering the inflatable retractor head when deflated.

Thus, according to one aspect of the present invention there is provided a tissue retractor (also referred to herein as the present device) for use in surgery, preferably minimally invasive surgery.

As used herein, the phrase “minimally invasive surgery” (also “endoscopic surgery”) refers to a surgical procedure in which the surgical workspace is not directly viewed or accessed by a surgeon. Laparoscopic surgery includes operations within the abdominal or pelvic cavities, whereas thoracoscopic surgery includes operations within the thoracic or chest cavity.

As used herein, the phrases “access site” or “tissue access site” refer to the incision in a tissue wall to provide access to a body cavity, while the phrases “access port” or “tissue access port” refer to a trocar, cannula or any other device which is positioned through a tissue access site to provide access to a body cavity.

The present device includes a shaft which is attached to an inflatable tissue retractor head positioned at a distal end thereof. The proximal end of the shaft is attachable to a handle via a releasable mechanism (e.g. quick snap) and as such, the handle is detachable from the shaft of the present tissue retractor.

The shaft can have any diameter and length suitable for minimally invasive surgery. Depending on the type of surgery, the configuration of the tissue retractor head and the size of access port used (inner diameter of cannula or trocar port), the shaft can be anywhere from 10 cm in length and 5 mm or less in diameter.

While reducing the present invention to practice, the present inventors fabricated a 5 mm alloy shaft which includes fluid conduits (for inflation of tissue retractor head) and yet maintains at least 80% of the axial rigidity of a normal 10 mm alloy shaft. Since laparoscopic tissue retraction requires a tool shaft that can efficiently transfer operator movements (at the handle) to the tissue retractor head, a shaft design having minimal deflection under side load is an advantage.

The distal end of the shaft is mechanically connected to the tissue retractor head via an immovable or hinged connection. Since the tissue retractor head is at least partially deployable via inflation, fluid conduits running through the shaft connect the tissue retractor head to a fluid source which can be situated within the handle or external to the device (in which case fluid ports are provided in the proximal end of the handle).

In order to enable fluid communication between a fluid source and the inflatable retractor head, the fluid conduit positioned within the shaft is connectable to a fluid conduit positionable within the handle via, for example, male-female plugs which include a seal (e.g. O-ring) or any similar mechanism. When the shaft is mounted on, and connected to, the handle, these plugs mate and form a fluid tight seal between the handle and shaft-positioned conduits.

The handle provides an interface with the surgeon and includes controls over deployment (e.g. inflation) and positioning of the tissue retractor head.

The inflatable tissue retractor head positioned at the distal end of the shaft is configured such that when inflated with a fluid (e.g. Air, CO₂ or Nitrogen gas, water, saline), it includes at least one extension which is sized and configured for enabling the tissue retractor head to hook over a tissue of, for example, an organ such as an intestine, a liver, a spleen, a lung, a uterus, a stomach, a kidney, a blood vessel such as an artery and connective tissue, fascia and the like. As used herein, the phrase “hook over” refers to the ability of the tissue retractor head to contact more than one side of a tissue when applied from the top thereof (i.e. the surface pointed in the direction of the surgical access incision). For example, in the case of an intestine, the tissue retractor head contacts more than 90 degrees of the organ circumference, preferably more than 180 degrees of the organ circumference, most preferably more than 270 degrees of the organ circumference when hooked over the organ.

The tissue retractor head can be configured as one or more interconnected extensions, or as one or more extensions projecting from a retractor head body. The extension or extensions typically angle away from the longitudinal plane of the handle by 20-60 degrees or by 20-60 degrees from the retractor head body depending on the shape and size of the extension and purpose of the device.

The deployed extension can be of any shape or size suitable for hooking over and optionally grasping the organ. Examples include, a prong, a hook, a claw and the like.

The functional shape of the deployed extension is dictated by one or more mechanisms. In the simplest configuration of the present retractor, the shape of the extension is largely controlled by inflation, i.e. the volume of inflation dictates the extent of deployment of the extension and its shape. In such cases, the extension is formed from a non-compliant balloon of a predetermined inflatable shape and volume.

For example, inflation of the tissue retractor head to a first predetermined volume can form an extension in a shape of a prong, while further inflation of the prong can form a hook. The transformation of the prong to a hook can take place by simply filling the same inflatable compartment with more fluid or by filling a second compartment which extends the prong into a hook. Alternatively, the shape and size of the extension can be governed by mechanical elements included within, or attached to, the inflatable tissue retractor head. For example, transformation between a prong and hook can take place by retracting a mechanical limiter off of a prong and further inflating the prong to form a hook. Further description of controlled and stepwise deployment of extensions is provided herein below.

Such controlled, stepwise deployment of the extension provides several advantages in grasping, moving and containing tissue. For example, partial deployment of a prong can be used for sweeping/raking tissue while full deployment as a hook enables grasping/lifting and moving of tissue. Conversely, deflation of a hook extension down to a prong can be used to more easily contain tissue following retraction.

The tissue retractor head is described in greater detail in WO2013144959 which is fully incorporated herein by reference.

The present device further includes a sheath for covering the tissue retractor head when deflated. The sheath is configured for facilitating passage of the tissue retractor head contained therein through a tissue incision (tissue access site) made for a 5 mm trocar port or cannula. In that respect, the sheath preferred embodiment is configured such that it is axially rigid and radially elastic. Axial rigidity enables the surgeon to advance the sheath (and contained retractor head) through the tissue incision site, while the radially elasticity allows the sheath to accommodate and tightly pack the deflated retractor head.

As is mentioned hereinabove, the detachable handle and the sheath of the present retractor configuration were designed specifically to enable access through narrow access ports (e.g. trocars with an internal diameter of 5 mm or less).

According to an aspect of some embodiments of the invention there is provided a tissue retractor device comprising an inflatable head configured at a distal end of a shaft, and a tensile element slidably received within one or more recesses of the head and configured to control and/or maintain an angular position of the head relative to the shaft. In some embodiments, the tensile element (for example in the form of a strap, string, cable or band) extends between the shaft and the head. Optionally, the tensile element is threaded through one or more holes in the inflatable head, for example slidably threaded through holes configured at webbing portions between the extensions of the head.

In some embodiments, attachment of the tensile element to the head allows the element to slide, at least to some extent, with respect to an outer surface of the head. In an example, the tensile element is wrapped around an extension, and threaded through holes configured in webbing portions of opposing sides of the extension. In some embodiments, sliding of the tensile element relative to the outer surface of the head reduces a risk of tear to the inflated head. Optionally, sliding is limited to an extent in which the element is stretched against the outer surface of the extension enough to provide for bending of the head relative to the shaft and/or to provide for maintaining an angular position of the head relative to the shaft.

In some embodiments, the tensile element is attached to the shaft by a hook like element. Optionally, the hook like element is axially movable within the shaft, and can be pulled proximally or advanced distally to pull or loosen the tensile element respectively. In some embodiments, two ends of the tensile element are attached to separate hook like elements that are axially movable with respect to the shaft and/or with respect to each other. Optionally, actuation of one of the hook like element (e.g. by pulling it in a proximal direction) pulls the end of the tensile element that is coupled to it, articulating the one or more finger like extensions of the head in a direction corresponding with the pulled end of the tensile element.

According to an aspect of some embodiments of the invention there is provided a retractor device in which the inflatable head is fixed at a linear position relative to the shaft. In some embodiments, the tensile element is configured to resist bending of the head relative to the shaft, to maintain a substantial 180 degree angle between the inflated head and the shaft. In some embodiments, the tensile element counter acts inflation forces to maintain the linear configuration.

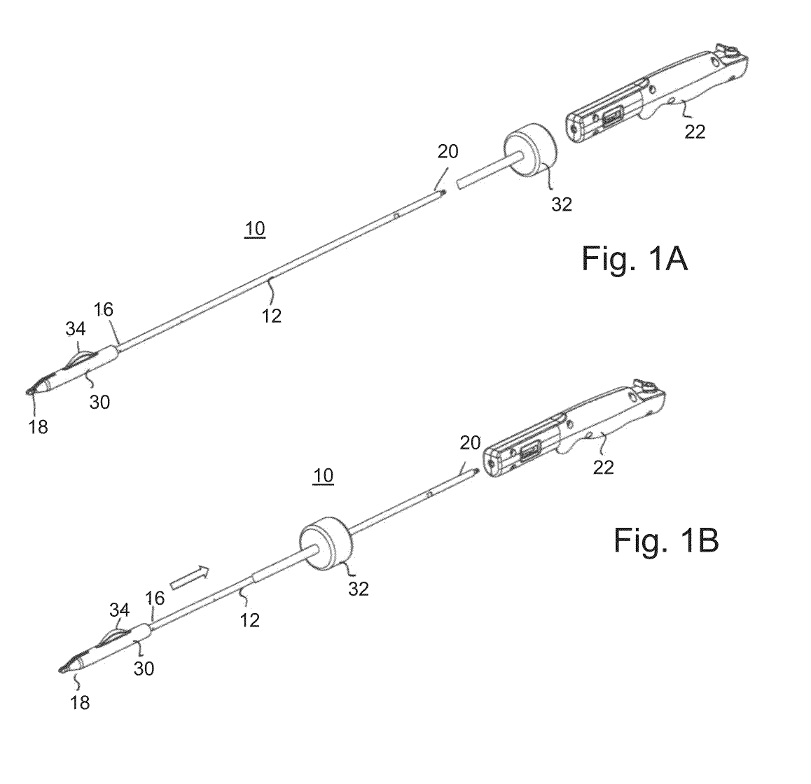

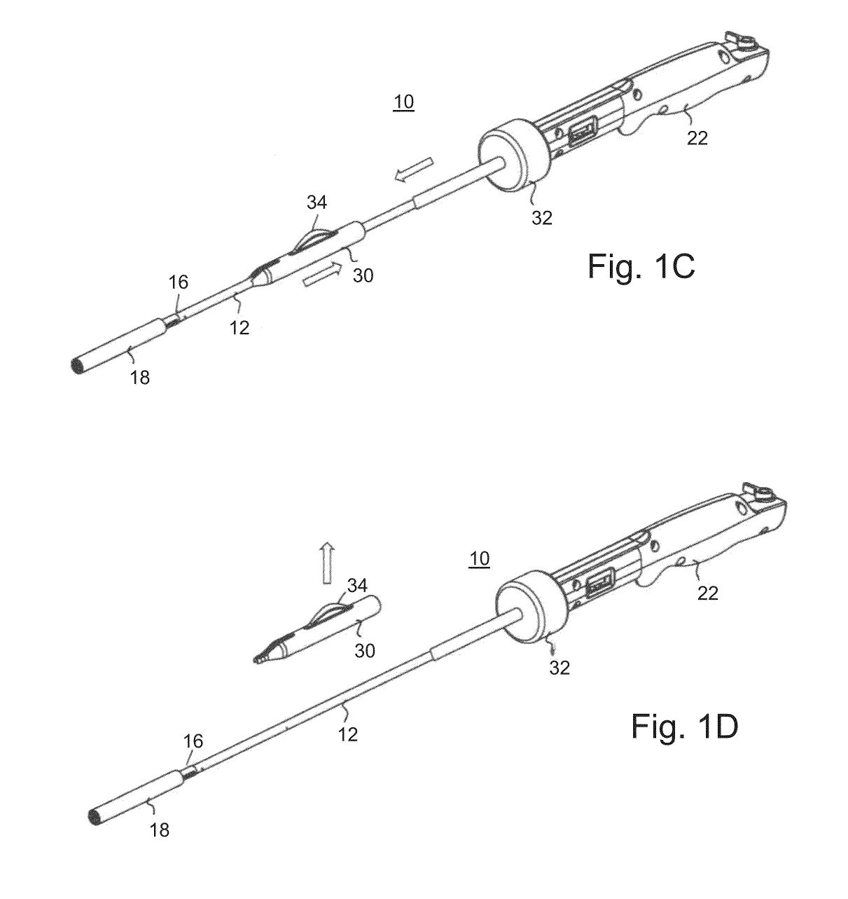

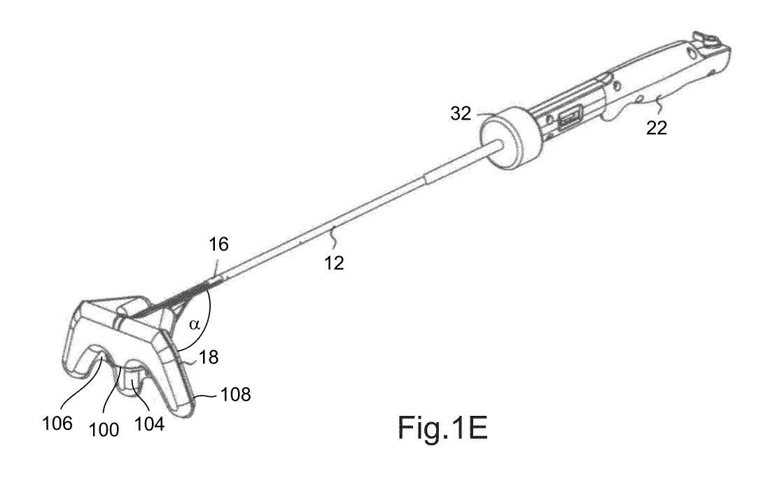

The construction of the present device, which is referred to herein as device 10 and its delivery into a body cavity are described in greater detail below with reference to FIGS. 1A-1E, 2, 3A-B, 4A-B and 5A-5E.

FIGS. 1A-E illustrate device 10 having a shaft 12 attached to a tissue retractor head 18 (also referred to herein as head 18) at a distal end 16 of shaft 12. Tissue retractor head 18 is inflatable to form the configurations described above and in WO2013144959.

Proximal end 20 of shaft 12 is attachable to a handle 22, the configuration of FIGS. 1A-B illustrate a configuration of device 10 in which shaft 12 is decoupled from handle 22 while FIGS. 1C-E illustrate a configuration of device 10 in which shaft 12 is coupled to handle 22. As is further described hereinbelow, such decoupling and coupling of handle 22 enables delivery of device 10 through a narrow access port.

Shaft 12 is configured as an elongated cylinder about 10-50 cm in length and 3-5.5 mm in diameter. Shaft 12 can be fabricated from a biocompatible alloy or polymer using techniques well known in the art. Shaft 12 is preferably hollow and/or includes fluid conduits that extend from a proximal end 20 to a distal end 16 thereof. The fluid conduits (not shown) enable inflation of tissue retractor head 18 with a fluid maintained under pressure in handle 22 or an external reservoir or pumping element (not shown).

Shaft 12 is preferably rigid but can include joints (e.g. hinges or swivel joints) for articulation of one portion of shaft 12 with respect to another. Such joints can be positioned close to distal end 16 or at a middle portion of shaft 12. Articulation around the joint can be controlled from handle 22 via a set of cables, geared transmission, threaded rods or axial plungers running within a lumen of shaft 12. It will be appreciated that in a case of a jointed shaft, the fluid conduits contained therein are preferably flexible to allow for articulation.

Handle 22 provides a user interface for controlling operation of device 10. Handle 22 enable a user to maneuver device 10 and control deployment of tissue retractor head 18. For example, handle 22 can include a release valve for releasing a pressurized fluid stored in handle 22 (or an external reservoir) through the fluid conduits and into tissue retractor head 18. Handle 22 can also include button for activating release of fluid from an inflated tissue retractor head 18 (via actuation of valves positioned within handle 22) and for actuating mechanical elements attached to, or disposed inside tissue retractor head 18, or for angling tissue retractor head 18 with respect to the longitudinal axis of shaft 12 as is further described in WO2013144959.

Device 10 further includes sheath 30 for covering and packing head 18 for delivery through a tissue access site.

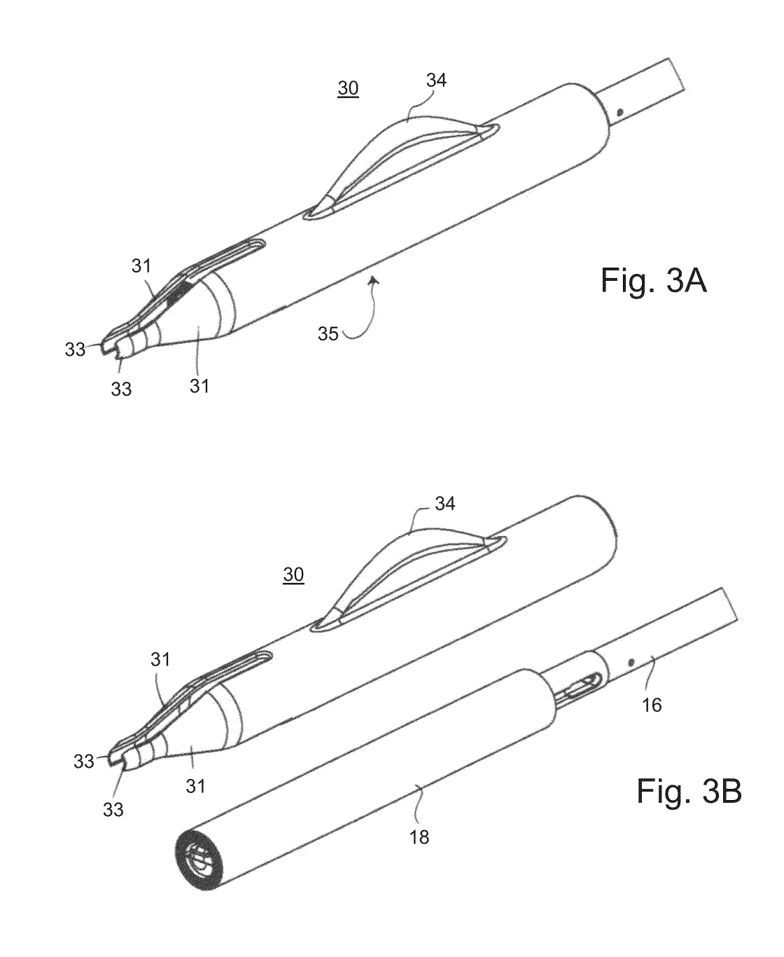

A typical tissue access site for a 5 mm trocar port can be up to 5 mm in diameter (can include multiple convergent incisions). When deflated, head 18 is roughly 9.5 mm in diameter and includes rolled layers of a pliable material. As such, delivery of head 18 through a trocar incision can be challenging. Sheath 30 covers a deflated head 18 and maintains the pliable folds in a tightly packed configuration. In addition, sheath 18 (which is described in more detail below with reference to FIGS. 3A-B) is configured for easily penetrating the tissue incision without buckling or bending.

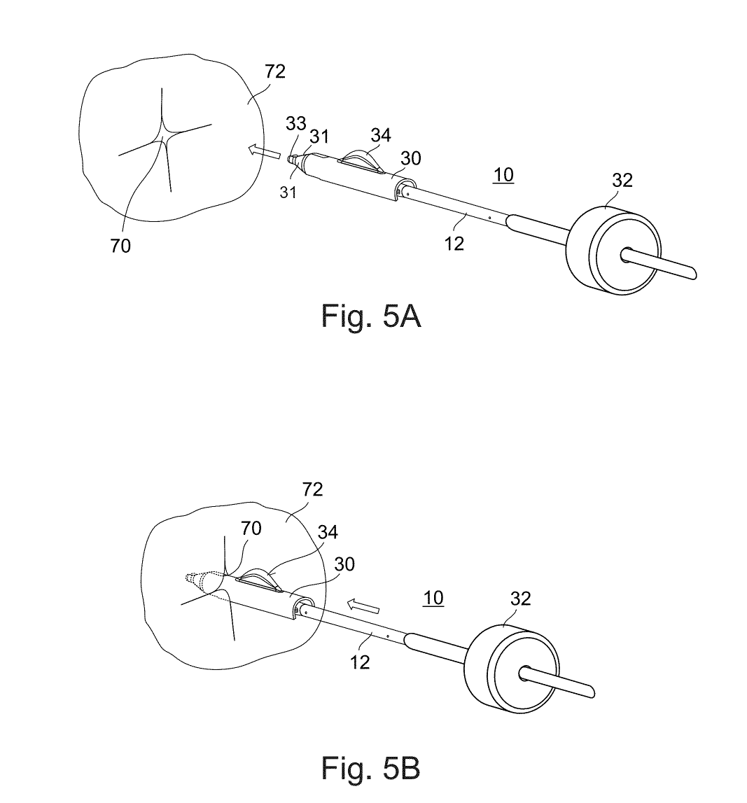

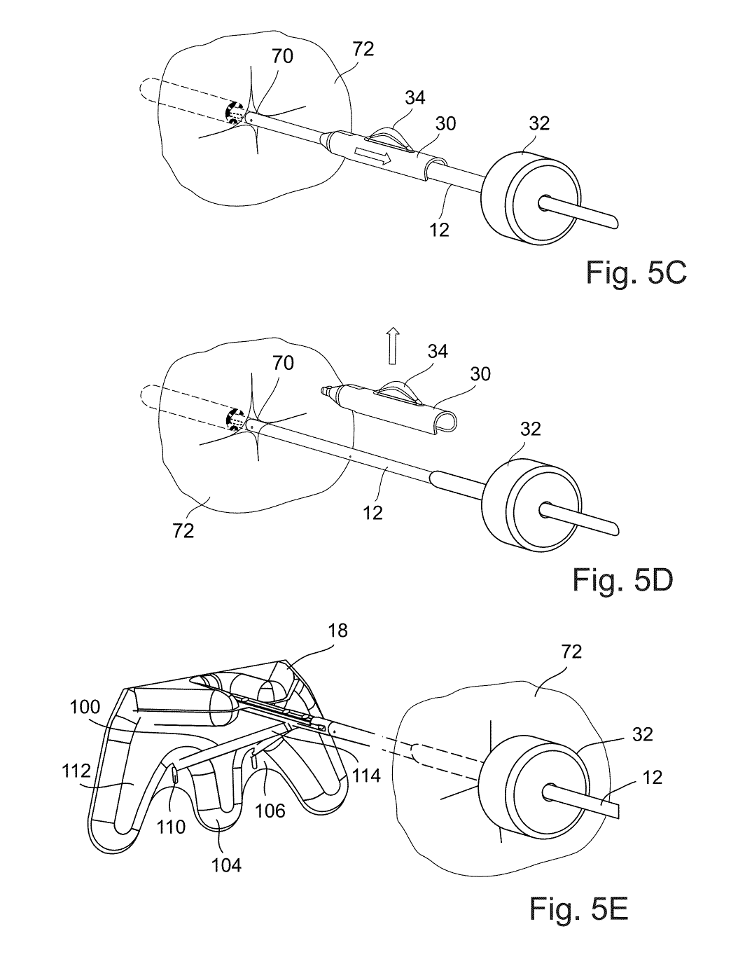

FIGS. 1A-E also illustrate the various device configurations throughout the delivery stages of head 18 into a body cavity. FIGS. 5A-E, which are described in greater detail below illustrate actual delivery through a tissue.

In FIG. 1A, shaft 12 is decoupled from handle 22 and a trocar 32 is positioned in between, sheath 30 covers and packs head 18. This is the initial configuration of device 10 prior to delivery. In FIG. 1B, trocar 32 is mounted on shaft 12 by threading the lumen of trocar 32 onto shaft 12 from proximal end 20—an end opposite to the typical insertion of trocars. Such mounting can be effected prior to or following delivery of sheath 30 (and enclosed head 18) through a tissue access site (incision). The former approach is preferred since it enables control over the angle of head 18 while also ensuring that head 18 can be pulled out through a larger trocar and re-inserted.

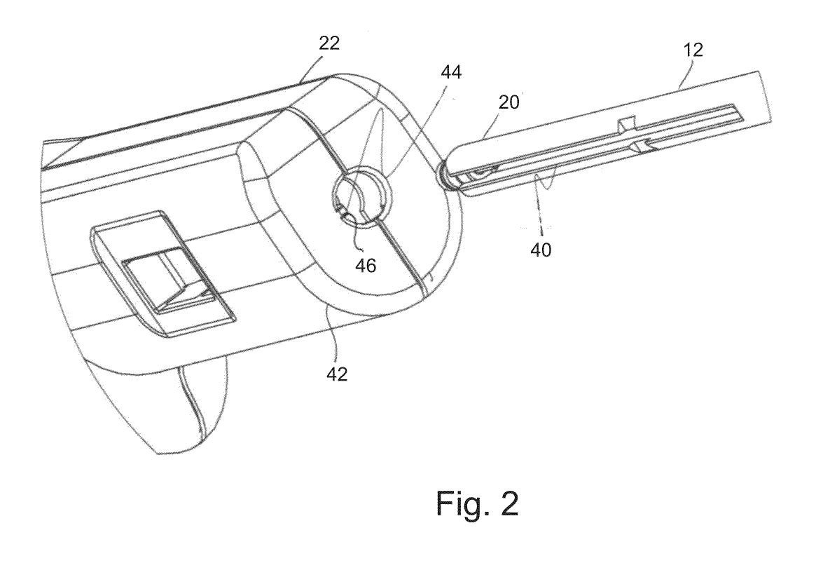

Once trocar 32 is mounted on the shaft, handle 22 can be coupled to shaft (FIG. 1C) as described in greater detail with reference to FIG. 2.

Sheath 30 includes a protrusion 34 (also referred to herein as stop 34) for preventing sheath 30 from completely going through the tissue access site. Thus, during delivery, sheath 30 lodges within the tissue access site and serves as a guide for advancing head 18 through sheath 30 and into the body cavity. As a result, sheath 30 slides back (in the proximal direction) along shaft 12 as is shown in FIG. 1C. Sheath 30 can also be pulled back if necessary by grasping stop 34.

Sheath 30 can then be removed (via stop 34) to allow relative movement between trocar 32 and shaft 12 (FIGS. 1D-E). FIG. 1E illustrates a deployed configuration of device 10 with head 18 inflated.

In some embodiments, for example as shown in FIG. 1E, the retractor device comprises a mechanical element for controlling angulation of inflatable head 18 relative to shaft 12. In some embodiments, the mechanical element is a tensile element 100, for example in the form of a strap, cable, band or string.

In some embodiments, tensile element 100 is coupled on one end to the inflatable head 18, and on the other end to shaft 12. Optionally, tensile element 100 is coupled to one or more of the finger like extensions 104 of the head. Optionally, for example as shown herein, the tensile element is wrapped around one of the extensions, such as the central extension. The tensile element may form a lasso like configuration, in which the element is looped around one or more of the finger like extensions.

In some embodiments, the tensile element is threaded through a webbing portion 106 between the extensions, as shown for example in FIG. 5E.

In some embodiments, tensile element 100 is configured for defining and/or maintaining an angle α between the head 18 and shaft 12. In some embodiments, angle a ranges between, for example, 20-180 degrees, such as 30 degrees, 60 degrees, 90 degrees, 120 degrees or intermediate, larger or smaller angles. In some embodiments, angle α (or an angle range) is selected according to the need, for example an angle range suitable for raking tissue, an angle range suitable for lifting tissue, an angle range suitable for pushing tissue, such as between 20-70 degrees, between 50-120 degrees, between 40-90 degrees, or intermediate, larger or smaller ranges.

In an example, element 100 can be pulled on to decrease angle α, and/or loosened in a distal direction to allow angle α to increase. In some embodiments, control of angulation of head 18 with respect to the shaft 12 using element 100 is actuated from handle 22.

In some embodiments, tensile element 100 provides a counter force that resists bending of the head relative to the shaft. In some embodiments, element 100 resists an external bending force that acts on the head, for example force applied to it by the tissue that was raked, pushed, hooked over and/or otherwise moved by the device. In some embodiments, element 100 resists a natural urge of the inflatable head, when inflated, to extend linearly with respect to the shaft.

In some embodiments, inflatable head 18 is formed of a soft, elastic material. Optionally, head 18 does not comprise any rigid components. In some embodiments, a seam 108 that outlines the head comprises a material more rigid than the material forming the body of the finger like extensions. A potential advantage of a more rigid seam may include reducing a risk of tearing of the member (for example due to over-inflation). Another potential advantage may include maintaining a shape (e.g contour) of the head, even when external forces act upon it. Alternatively, in some embodiments, head 18 is seamless.

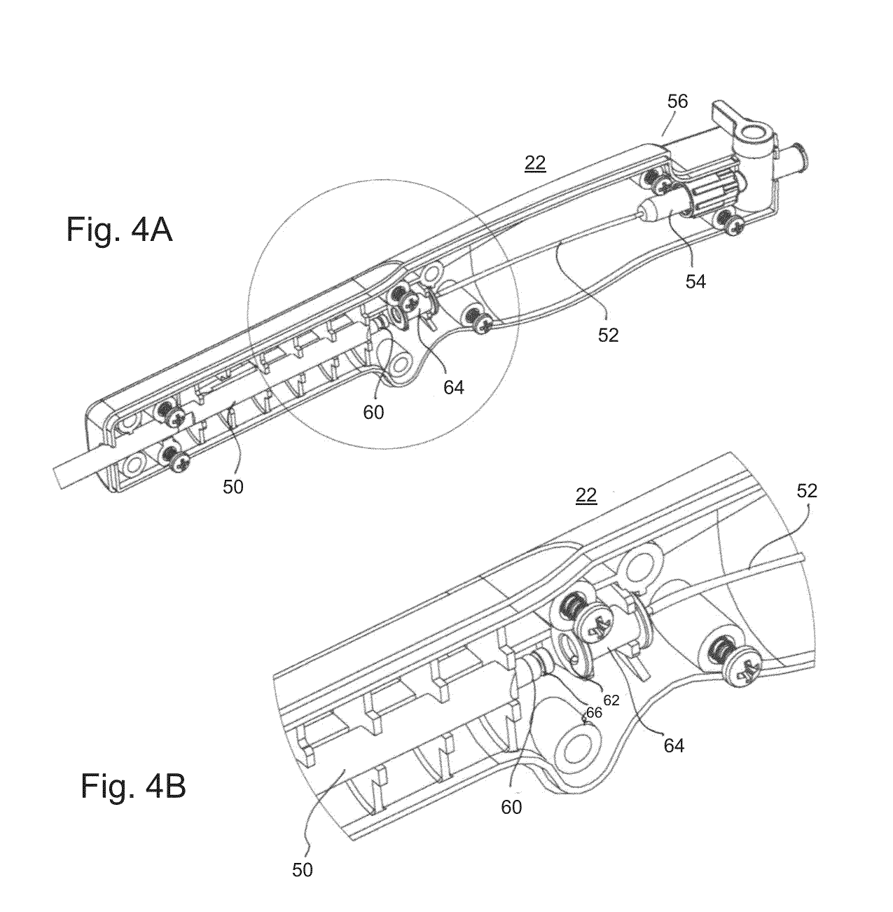

FIG. 2 illustrates one embodiment of a mechanism suitable for coupling/decoupling shaft 12 and handle 22. In this embodiment, proximal end 20 of shaft 12 includes a slit 40, while distal end 42 of handle 22 includes an opening 44 provided with a guiding pin 46. Slit 40 in shaft 12 engages guiding pin 46 within opening 44 to snap together shaft 12 and handle 22. As is further described hereinunder with respect to FIGS. 4A-B, this coupling also aligns the fluid conduits of shaft 12 and handle 22 for engagement. Alternative coupling mechanism can include a male needle and female septum or the like.

FIGS. 3A-B illustrate sheath 30 which functions in covering and packing head 18 and for enabling delivery thereof through the tissue access site as described above.

Sheath 30 is typically c-shaped (in cross section) and is radially flexible with an inner diameter of about 9 mm to hold head 18 folded under compressive forces. The C-shape enables removal of sheath 30 from head 18 through slit 35 (as shown in FIG. 1D). The length of sheath 30 can be 80-100 mm while distal protrusions 31 (2-4) can be 20-50 mm in length and conical shaped down to 2-3 mm at tip 33 (penetrating end). Sheath 30 can be fabricated from a biocompatible polymer such as polycarbonate or any other suitable material which provides the requisite radial elasticity and longitudinal (axial) rigidity.

FIGS. 4A-B illustrate one embodiment of a fluid connection between conduits of shaft 12 and handle 22.

Shaft 12 includes conduit 50 which runs along the length of shaft 12 from proximal end 20 to distal end 16 and into head 18. Handle 22 includes conduit 52 which is connected to a fluid inflation port 54 at proximal end 56 of handle 22 (as shown) or to a fluid source provided within handle 22 (not shown).

As is shown in the magnified view of FIG. 4B, conduit 50 includes a male connector 60 which engages an inner lumen 62 of female connector 64 attached to conduit 52. Male connector 60 includes an O-ring 66 for providing a fluid tight seal once connectors 60 and 66 are engaged.

Delivery of device 10 through a tissue access site 70 of a tissue wall 72 surrounding a body cavity and deployment of head 18 within the cavity is shown in FIGS. 5A-E.

FIG. 5A illustrates shaft 12 of device 10 with trocar 32 mounted on shaft 12 and sheath 30 positioned over head 18. Handle 22 is decoupled from shaft 12 at this stage to enable mounting of trocar 32 (which is not a part of device 10). Once trocar 32 is mounted handle 22 (not shown) can be coupled to shaft 12 as described above. Sheath 30 is advanced through tissue access site 70 (FIG. 5B) until stop 34 engages the outer surface of tissue wall 72, at this stage sheath 30 lodges in tissue access site 70, further advancing of shaft 12 into the body cavity frees head 18 from sheath 30 and positions head 18 within the body cavity (FIG. 5C). Sheath 30 is removed and trocar 32 is advanced into tissue access site 70 (FIG. 5D). Head 18 is then deployed (inflated and angled) using controls provided on handle 22 and device 10 is operated to retract intra-cavity tissue in the manner described in WO2013144959.

In FIG. 5E, head 18 is angled relative to shaft 12, for example being perpendicularly oriented relative to the shaft. Tensile element 100 extends between shaft 12 and head 18, and in this example is shown to be threaded through designated holes 110 in webbing portions 106 connecting the finger like extensions 104.

In some embodiments, tensile element 100 is free to move within hole 110, for example to slide back and forth within the hole. Optionally, movement of the tensile element is restricted, for example at a coupling point 114 between the tensile element and the shaft 12. Optionally, tensile element 100 rests loosely against the finger like extension. A potential advantage of a tensile element that is free to slide against the surface of the finger like extension may include reducing a risk of tear of the inflated extension. Another potential advantage of a tensile element that is not fixedly attached to the head (e.g. an element coupled to the head by an adjustable attachment, for example a tensile element threadedly received within a hole) may include avoiding a rigid attachment, for example comprising a suture and/or other attachment which may damage (e.g. scratch) the tissue.

Additionally or alternatively, in some embodiments, tensile element 100 may be coupled to other portions of the head, for example mounted to (e.g. by being glued to) a proximally facing face 112 of head 18. Optionally, the tensile element is slidably threaded through loops or straps mounted on the face of the head.

In some embodiments, a coupling between tensile element 100 and head 18 is selected to provide stability. In the exemplary configuration shown herein, tensile element 100 is coupled to head 18 at opposing sides of the central finger like extension 104.

In some embodiments, webbing portion 106 is shaped and/or sized to allow movement, at least to some extent, of the finger like extensions relative to each other. Optionally, webbing portion 106 is formed of a flexible sheet of material.

Optionally, webbing portion 106 is formed of a tear resistant material, for example to reduce a risk of tear due to movement of the tensile element within hole 110. In an exemplary embodiment, webbing portion 106 comprises one or more layered sheets, optionally welded. A thickness of a sheet may range between, for example, 0.05 mm to 0.5 mm

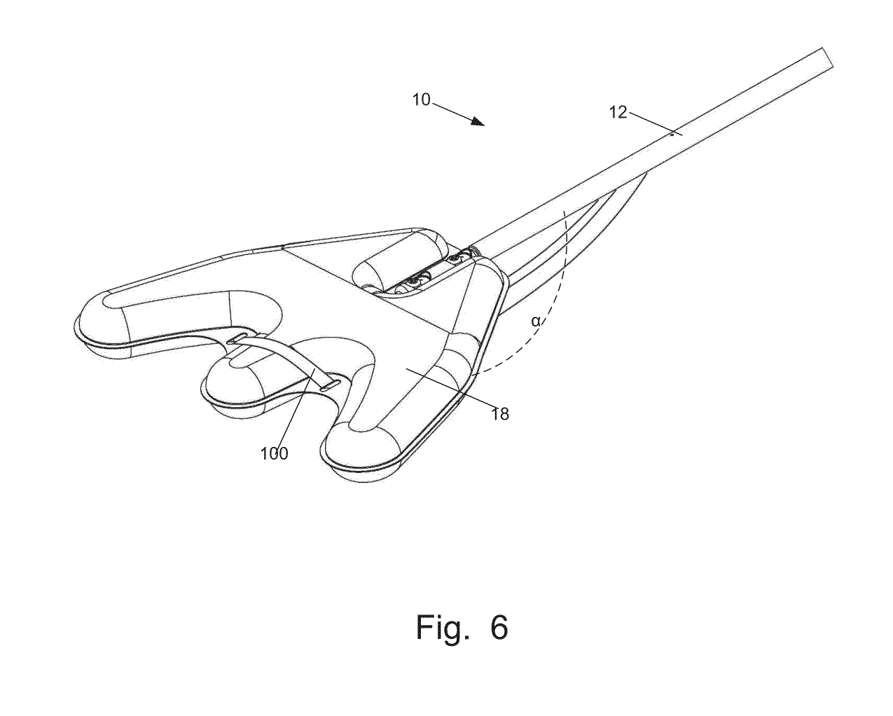

FIG. 6 shows a distal portion of device 10, in which inflatable head 18 is configured in a linear position relative to shaft 12. In some embodiments, tensile element 100, for example in the form of a strap, maintains the linear position (in which angle α is substantially 180 degrees) by resisting bending of the head. In some embodiments, inflation of head 18 increases a thickness of the head which may cause at least a part of the head to rise, optionally producing an angle higher than 180 degrees relative to the shaft, and tensile element 100 counter acts the inflation forces to maintain the head at a flat configuration relative to the shaft.

In some embodiments, in the linear configuration, device 10 is used for pushing tissue, for example in a distal direction. Additionally or alternatively, in the linear configuration, device 10 is used for lifting tissue.

In an exemplary embodiment, the head comprises only one finger like extension. In some embodiments, a shape and/or size of the single finger like extension are selected according to the intended use of the device, for example, in a device configured for engaging a liver (such as to rake, lift, push, and/or otherwise move the liver) the extension may comprise a rounded spoon-like shape.

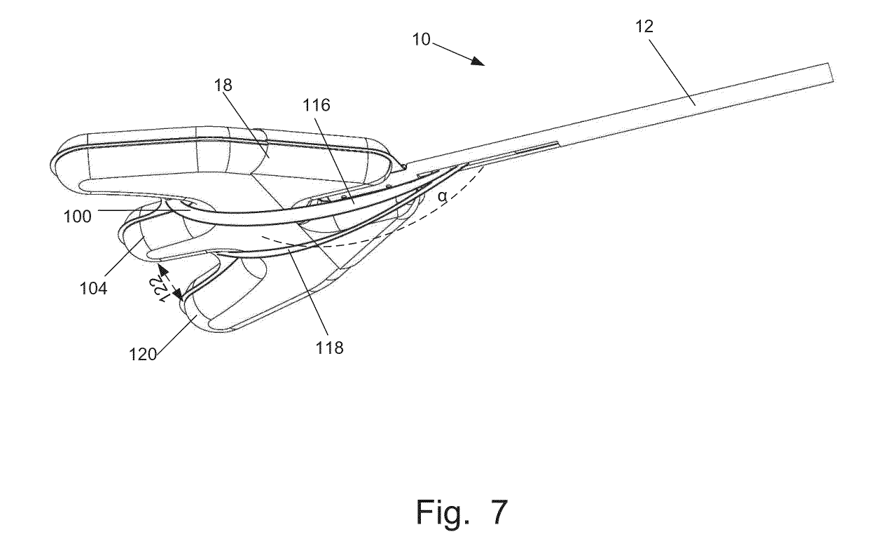

FIG. 7 shows a distal portion of device 10, in which inflatable head 18 is configured in a linear position relative to shaft 12, shown from a direction opposite to the one shown in FIG. 6.

In some embodiments, for example as shown herein, tensile element 100 (for example in the form of a strap) is wrapped around one or more of the finger like extensions such as extension 104. In some embodiments, the free ends such as 116 and 118 of the tensile element 100 extend to shaft 12. Optionally, the ends are received within one or more slots 118 in shaft 12.

In some embodiments, the device is configured to be maintained in a constant linear configuration (angle α of approximately 180 degrees). Optionally, the constant angle is maintained by tensile element 100, for example by tensioning the element enough to resist bending of the inflatable head 18 relative to shaft 12.

Alternatively, in some embodiments, tensile element 100 provides for angulation of head 18 relative to shaft 12. Optionally, angulation is within the range of 20-180 degrees. Additionally or alternatively, angulation is within the range of 20-340 degrees.

In some embodiments, head 18 is configured to grasp tissue in between the finger like extensions, for example in a tweezer-like motion. Optionally, one or more of the finger like extensions can be moved relative to each other. For example, central extension 104 can be moved towards side extension 120, such that a distance 122 between the extensions is reduced. In some embodiments, control of the grasping tweezer-like movement is provided by tensile element 100.

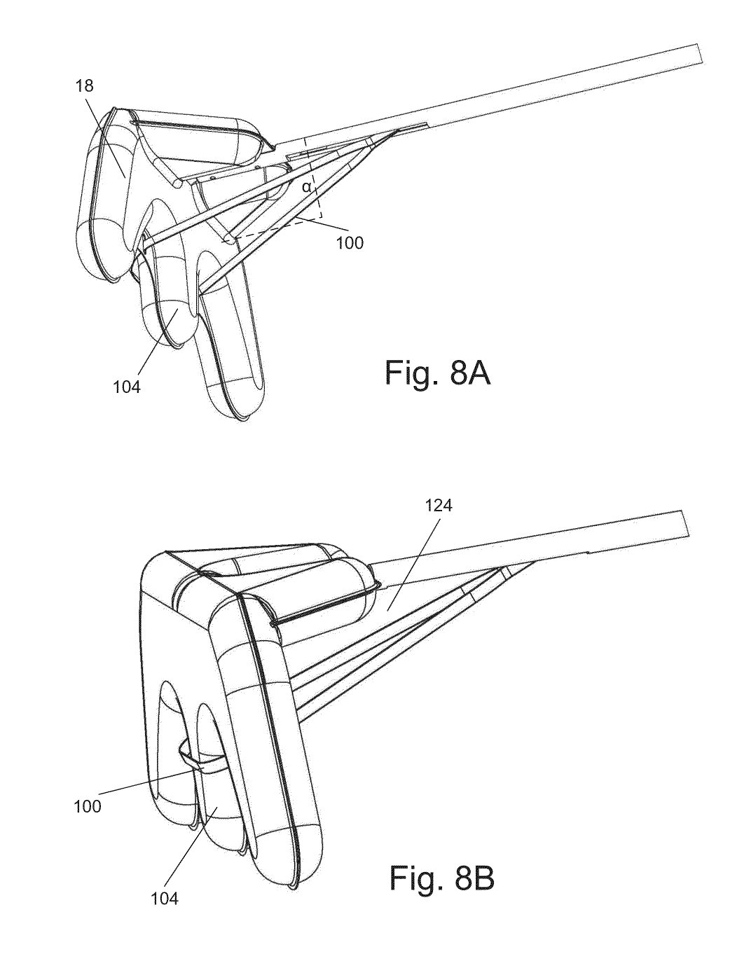

FIGS. 8A-B show a back isometric view and a front isometric view showing the inflatable retractor head bent at an angle relative to the shaft.

Optionally, for example as shown herein, tensile element 100 (for example in the form of a strap) is loosely wrapped around an extension, such as central extension 104.

In some embodiments, when head 18 is angulated with respect to the shaft, for example being substantially perpendicular to the shaft as shown herein, an opening 124 (shown from a side view in FIG. 8B) is formed between the shaft 12, head 18 and tensile element 100. Optionally, the opening is a triangular window in which the tensile element, which extends between the shaft and the head, forms the hypotenuse of the triangle.

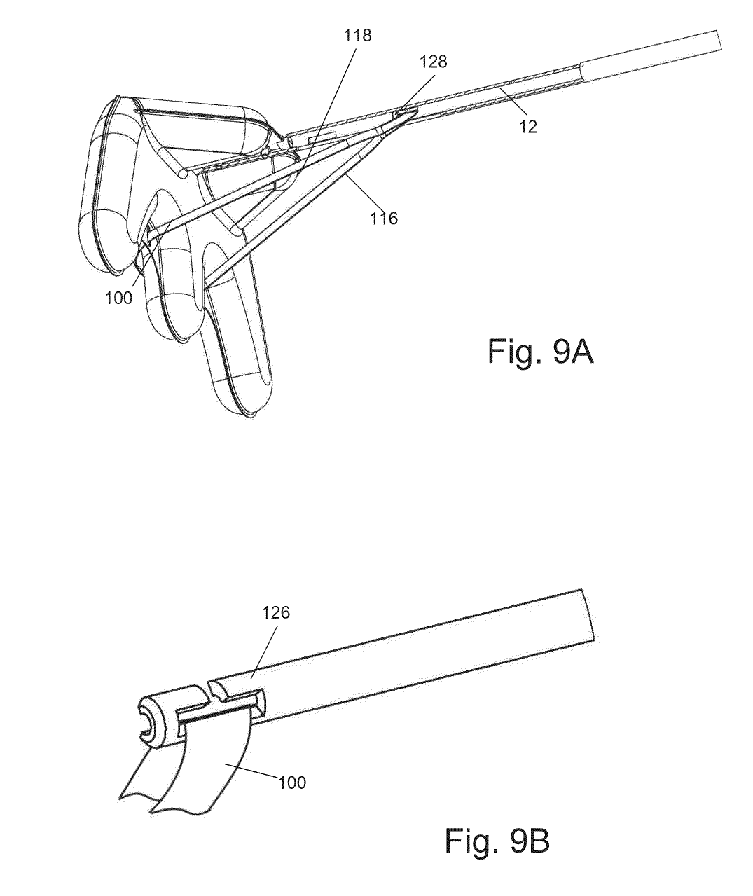

FIGS. 9A-B illustrate an exemplary coupling configuration between the tensile element and the shaft of the device, according to some embodiments of the invention.

In some embodiments, tensile element 100, for example the free ends 116 and 118 of the element are received within one or more slots 128 of shaft 12. In some embodiments, the tensile element hooks onto an element 126 that is axially moveable within shaft 12, for example to enable pulling and/or loosening the tensile element.

Optionally, element 126 is shaped to trap the tensile element within it, keep the tensile element attached to the shaft so that even in a situation in which tensile element 100 is torn or otherwise damaged, the tensile element can be entirely retracted from the body.

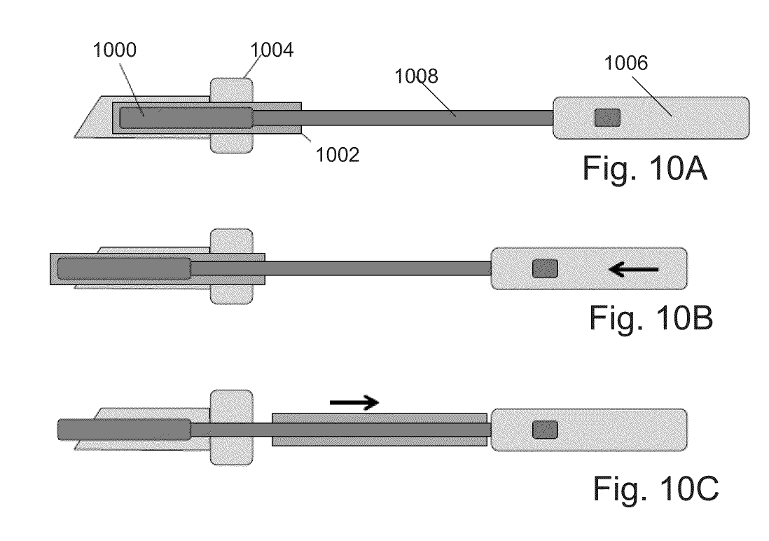

FIGS. 10A-C illustrate an exemplary structure of the device in which a sheath 1002 is axially slidable in the proximal and/or distal directions on shaft 1008. In some embodiments, sheath 1002 covers the inflatable retractor head 1000 in a deflated state, as shown for example in FIG. 10A, and can be moved proximally in the direction of handle 1006 to expose the deflated head 1000, as shown for example in FIG. 10C.

In some embodiments, sheath 1002 (and the deflated head contained therein) is insertable into a lumen of trocar 1004, providing for advancing the deflated head distally within the trocar while the head is protected within the sheath, as shown for example in FIG. 10B. In some embodiments, once the head is advanced to a distal opening of the trocar lumen, sheath 1002 is pulled in the proximal direction, exiting trocar 1004.

Although the present invention was described with reference to a tissue retractor device having an inflatable retractor head, it should be noted that the handle-shaft coupling-decoupling configuration and delivery approach described herein, as well as the sheath configured for delivery through a tissue access site can be used with other surgical devices delivered through a tissue access site and operated through a tissue access port.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A tissue retractor device comprising:
an inflatable tissue retractor head attached to a distal end of a shaft, said inflatable retractor head comprising one or more finger-like extensions shaped and sized to move tissue within a body cavity, and
a tensile element coupled to said inflatable tissue retractor head, said tensile element slidably received within one or more recesses configured in said head.

2. The device according to claim 1, wherein said tensile element is configured to resist bending of said head relative to said shaft from a set position.

3. The device according to claim 1, wherein said set position comprises an angulated position of said head relative to the said shaft.

4. The device according to claim 3, wherein an angle between said head and said shaft is between 20-180 degrees.

5. The device according to claim 1, wherein said tensile element controls angulation of said head relative to said shaft.

6. The device according to claim 1, wherein said one or more recesses of said head are located at one or more webbing portions connecting between said finger-like extensions, and said tensile element is threadedly received within said one or more recesses.

7. The device according to claim 1, wherein said tensile element extends between said head and said shaft.

8. The device according to claim 7, wherein said tensile element is attached to said shaft by a hook like element, said hook like element axially moveable within said shaft.

9. The device according to claim 8, wherein each end of the tensile element is attached to a separate hook like element, said elements axially movable with respect to said shaft and with respect to each other to articulate said head.

10. The device according to claim 1, wherein said tensile element is configured to slide with respect to an outer surface of said head to a limited extent that reduces a risk of tear to said head when inflated, while still providing for control of angulation using said tensile element.

11. The device according to claim 1, wherein pulling on said tensile element reduces said angle between said head and said shaft, and loosening said tensile element allows said angle to increase.

12. The device according to claim 1, wherein said tensile element is strong enough to resist a natural urge of said head, when inflated, to extend linearly with respect to said shaft.

13. The device according to claim 1, wherein said set position comprises a linear position of said head with respect said shaft, in which said head is positioned substantially at a 180 degree angle relative to said shaft.

14. The device according to claim 1, wherein said tensile element in the form of a strap, string, cable or band.

15. The device according to claim 1, wherein said tensile element is wrapped around at least one finger-like extension, forming a lasso-like configuration.

16. The device according to claim 1, wherein said head comprises two recesses through which said tensile element extends, said recesses symmetrically arranged with respect to said head.

17. The device according to claim 1, wherein said tensile element is external to said inflatable retractor head.

18. The device according to claim 1, further comprising a sheath shaped and sized to cover said inflatable head in a deflated state, said sheath shaped and sized to slidably fit within a lumen of a trocar.

19. A tissue retractor device comprising:
an inflatable tissue retractor head attached to a distal end of a shaft, said inflatable retractor head comprising one or more finger-like extensions shaped and sized to move tissue within a body cavity, and
a tensile element coupled to said inflatable tissue retractor head, said tensile element resisting bending of said inflatable tissue retractor head relative to said shaft to maintain said head at a substantially linear configuration relative to said shaft.

20. A tissue retractor device comprising:
(a) an inflatable tissue retractor head being attached to a distal end of a shaft having a conduit extending from a proximal end of said shaft to said inflatable tissue retractor; and
(b) a handle being detachably attached to said proximal end of said shaft, said handle including a port for communicating an inflation fluid through said handle and said conduit to said inflatable tissue retractor when said handle is attached to said proximal end of said shaft.

21. The device of claim 20, wherein said port is in fluid communication with a lumen in said handle, said lumen being capable of sealing with said conduit when said handle is attached to said proximal end of said shaft.

22. The device of claim 20, wherein said inflatable tissue retractor head is configured such that when inflated with a fluid, said tissue retractor head forms a configuration which includes a plurality of finger-like extensions.

23. The device of claim 20, further comprising a mechanism for angling a portion of said tissue retractor head with respect to said handle during or following inflation of said tissue retractor head.

24. The device of claim 20, further comprising a sheath for covering said tissue retractor head when deflated, said cover being configured for delivery through a tissue incision.

25. The device of claim 24, wherein said sheath is axially rigid to enable penetration through said incision and radially flexible to enable covering of inflatable tissue retractor head when deflated.

26. A tissue retractor device comprising:
(a) an inflatable tissue retractor head being attached to handle through a shaft; and
(b) a sheath for covering said tissue retractor head when deflated, said sheath being configured for delivery through an incision.

27. The device of claim 26, wherein said sheath is axially rigid to enable penetration through said incision and radially flexible to enable covering of inflatable tissue retractor head when deflated.

28. The device of claim 26, wherein said sheath include an external protrusion for preventing said sheath from penetrating completely through said incision.

29. A method of positioning a laparoscopic device in a body cavity, the method comprising:
(a) delivering a working head of the laparoscopic device through an incision and into the body cavity using a device shaft attached thereto;
(b) sliding a trocar over said shaft; and
(c) attaching a handle of the laparoscopic device to said shaft.

30. The method of claim 29, further comprising inserting said trocar into said incision following (b) or (c).

31. The method of claim 29, wherein said working head is an inflatable tissue retractor head.

32. The method of claim 31, wherein (a) is effected by packing said tissue retractor head into a sheath configured for delivery through said incision.

33. The method of claim 32, wherein said sheath is axially rigid to enable penetration through said incision and radially flexible to enable covering of inflatable tissue retractor head when deflated.

34. The method of claim 32, further comprising (d) removing said sheath and inflating said inflatable tissue retractor head from said handle.
